(19) United States
(12) Patent Application Publication
(10) Pub. No.: US 2024/0008744 A1
(43) Pub. Date: Jan. 11, 2024

(54) DIAPLASIS DETECTION APPARATUS AND SYSTEM, AND DIAPLASIS DATA PROCESSING METHOD AND APPARATUS

(71) Applicant: BOE Technology Group Co., Ltd., Beijing (CN)

(72) Inventors: Bing ZHAO, Beijing (CN); Yuan WANG, Beijing (CN)

(21) Appl. No.: 17/312,236

(22) PCT Filed: Dec. 30, 2020
(86) PCT No.: PCT/CN2020/141329

§ 371 Date: Sep. 30, 2021

(30) Foreign Application Priority Data
Mar. 24, 2020 (CN) 202010211325.X

(51) Int. Cl. A61B 5/00; A61B 42/10

(52) U.S. Cl. CPC A61B 5/0053; A61B 5/6826; A61B 42/10

(57) ABSTRACT

A diaplasis detection apparatus, a diaplasis detection system, a diaplasis data processing method and a diaplasis data processing apparatus are provided. The diaplasis detection apparatus includes: a glove body, where a plurality of positions on the glove body is provided with pressure sensors respectively, and the plurality of positions include positions on the glove body corresponding to a position on a palm used to fix a force applied part and a position on the palm used to perform traction and diaplasis on the force applied part to when the diaplasis is performed by using the glove body; a data collection circuit connected to each pressure sensor and configured to collect a pressure signal, and a transmission circuit configured to acquire the pressure signal and transmit the pressure signal to a processing apparatus.

### CROSS-REFERENCE TO RELATED APPLICATIONS

The present disclosure claims a priority to Chinese Patent Application No. 202010211325.X filed on Mar. 24, 2020 in China, the disclosure of which is incorporated in its entirety by reference herein.

### TECHNICAL FIELD

The present disclosure relates to the field of detection technology, and more particularly, to a diaplasis detection apparatus, a diaplasis detection system, a diaplasis data processing method and a diaplasis data processing apparatus.

### BACKGROUND

A closed diaplasis surgery has such unique advantages as non-invasive in a process of fracture reduction of a patient, and it is able to reduce surgical risks brought by invasive surgery and avoid high surgical costs. However, a lot of clinical practices are required when performing the effective closed diaplasis surgery, and are taught by a master in a hand-to-hand teaching manner. Further, force applied points and force applied parameters when performing the closed diaplasis surgery cannot be accurately determined, which is not convenient for subsequent case guidance, robot-arm implementation, medical course, etc.

### SUMMARY

An object of the technical solution of the present disclosure is to provide a diaplasis detection apparatus and a diaplasis detection system, a diaplasis data processing method and a diaplasis data processing apparatus, so as to solve the problem in the prior art that the force applied points and the force applied parameters when the diaplasis is performed cannot be accurately collected.

A diaplasis detection apparatus is provided, including: a glove body capable of being worn on a hand of a force applying person, where a plurality of positions on the glove body is provided with pressure sensors respectively, and the plurality of positions includes positions on the glove body corresponding to a position on a palm used to fix a force applied part and a position on the palm used to perform traction and diaplasis on the force applied part when the diaplasis is performed by using the glove body; a data collection module connected to each pressure sensor and configured to collect a pressure signal detected by the pressure sensor; and a transmission module configured to acquire the pressure signal and transmit the pressure signal to a processing apparatus.

Optionally, the diaplasis detection apparatus further includes a mounting housing separate from the glove body, and the data collection module and the transmission module are fixed on the mounting housing.

Optionally, the plurality of positions includes the positions on the glove body corresponding to a hypothenar position on a medial side of the palm, finger pulps of five fingers of the palm, a thenar position on the palm, a lateral position at a first joint of an index finger close to a thumb, a lateral position at a second joint of the index finger close to the thumb, a position at the first joint of the index finger on a palmar surface and a position at the second joint of the index finger on the palmar surface.

Optionally, the data collection module includes a plurality of data collection channels, each data collection channel is configured to acquire the pressure signal detected by one corresponding pressure sensor, and the transmission module is configured to acquire the pressure signal on each data collection channel and transmit the pressure signal on the data collection channel to the processing apparatus.

Optionally, the data collection module includes a data processing chip having general-purpose input/output (GPIO) ports, and each GPIO port corresponds to one data collection channel.

Optionally, the transmission module includes a Bluetooth transmission module or a Wireless Fidelity (Wi-Fi) transmission module.

Optionally, the number of glove bodies is two, the plurality of positions on each glove body is provided with the pressure sensors respectively, and the data collection module is connected to each pressure sensor on the two glove bodies.

A diaplasis data processing method for performing diaplasis data processing by using the above-mentioned diaplasis detection apparatus is further provided, including: acquiring the pressure signal transmitted by the transmission module; and acquiring pressure data at the plurality of position points when the diaplasis is performed by using the glove body in accordance with the pressure signal; where one pressure sensor corresponds to one position point.

Optionally, the diaplasis data processing method further includes: establishing an association relationship between the pressure data and pre-stored user information; and storing the pressure data and the association relationship.

Optionally, the diaplasis data processing method further includes displaying a time-changing waveform of the pressure data at one of the position point when the diaplasis is performed by using the glove body.

Optionally, the diaplasis data processing method further includes displaying a magnitude relationship among the pressure data at the position points in a comparison manner when the diaplasis is performed by using the glove body.

A diaplasis data processing apparatus for performing diaplasis data processing by using the above-mentioned diaplasis detection apparatus is further provided, including: a signal acquisition module, configured to acquire the pressure signal transmitted by the transmission module; and an analysis module, configured to acquire pressure data at the plurality of position points when the diaplasis is performed by using the glove body in accordance with the pressure signal; where one pressure sensor corresponds to one position point.

Optionally, the diaplasis data processing apparatus further includes: a processing module, configured to establish an association relationship between the pressure data and pre-stored user information; and a storage module, configured to store the pressure data and the association relationship.

Optionally, the diaplasis data processing apparatus further includes a first display module, configured to display a time-changing waveform of the pressure data at one of the position points when the diaplasis is performed by using the glove body.

Optionally, the diaplasis data processing apparatus further includes a second display module, configured to display a magnitude relationship among the pressure data at the position points in a comparison manner when the diaplasis is performed by using the glove body.

A diaplasis detection system including the above-mentioned diaplasis detection apparatus and the above-mentioned diaplasis data processing apparatus is further provided.

### BRIEF DESCRIPTION OF THE DRAWINGS

In order to illustrate the embodiments of the present disclosure or the technical solutions in the related art in a clearer manner, a brief description of the drawings required to be used in the description of the embodiments will be given below. Obviously, the following drawings merely relate to some embodiments of the present disclosure, and based on these drawings, a person of ordinary skill in the art may obtain other drawings without any creative effort.

FIG. 1 is a schematic structural diagram of a diaplasis detection apparatus according to the embodiment of the present disclosure;

FIG. 2 is a schematic structural diagram of relative positions of various piezoelectric thin film sensors 11 in the diaplasis detection apparatus according to the embodiment of the present disclosure;

FIG. 3 is a schematic diagram of a connection relationship between the piezoelectric film sensor and a data collection module in the diaplasis detection apparatus according to the embodiment of the present disclosure;

FIG. 4 is a schematic flowchart of a diaplasis data processing method according to the embodiment of the present disclosure;

FIG. 5 is a schematic flowchart of a diaplasis data processing apparatus according to the embodiment of the present disclosure;

FIG. 6 is a schematic structural diagram of a diaplasis detection system according to the embodiment of the present disclosure;

FIG. 7 is a schematic diagram of main processes of the system according to the embodiment of the present disclosure; and

FIG. 8 is a schematic diagram of a display state of an upper-computer interface according to the embodiment of the present disclosure.

### DETAILED DESCRIPTION

In order to make the technical problems to be solved, technical solutions and advantages of the present disclosure clear, a detailed description will be given below with reference to the accompanying drawings and specific embodiments.

In order to solve the problem in the prior art that force applied points and force applied parameters when the diaplasis is performed cannot be accurately collected, a diaplasis detection apparatus, a diaplasis detection apparatus system, a diaplasis data processing method and a diaplasis data processing apparatus are provided in the embodiments of the present disclosure. A glove body for wearing during an implementation of closed fracture reduction is provided, piezoelectric thin film sensors capable of detecting pressures during the implementation of closed fracture reduction are arranged on the glove body, a data collection module is configured to collect a pressure signal detected by each piezoelectric thin film sensor, and a wireless transmission module is configured to transmit the pressure signal to a processing apparatus, so that force applied points and force applied parameters when the diaplasis is performed can be accurately acquired, which is convenient for subsequent case guidance, robot-arm implementation, medical course, etc.

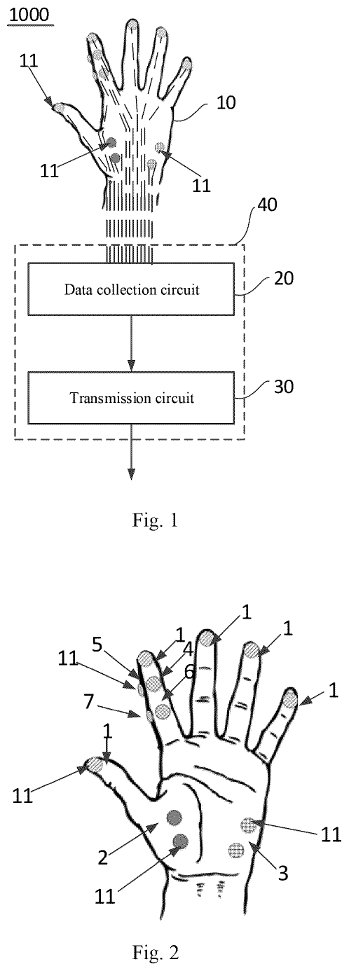

In the embodiments of the present disclosure, as shown in FIG. 1, a diaplasis detection apparatus 1000 includes: a glove body 10 capable of being worn on a hand of a force applying person, where a plurality of positions on the glove body 10 is provided with pressure sensors 11 respectively, and the plurality of positions includes positions on the glove body corresponding to a position on a palm used to fix a force applied part and a position on the palm used to perform traction and diaplasis on the force applied part when the diaplasis is performed by using the glove body; a data collection module 20 connected to each pressure sensor 11 and configured to collect a pressure signal detected by the pressure sensor; and a transmission module 30 configured to acquire the pressure signal and transmit the pressure signal to a processing apparatus.

Optionally, the glove body 10 may be made of silicone or a flexible cloth. The pressure sensor 11 may be enclosed in an interior of the glove body 10 or on an inner surface of the glove body 10, and may sense a pressure applied at a corresponding position during the implementation of closed fracture reduction and convert a pressure value into an electrical signal.

Each pressure sensor 11 on the glove body 10 corresponds to one force applied point, and the pressure sensors 11 at different positions detect pressure signals at corresponding positions respectively.

In the embodiments of the present disclosure, optionally, the pressure sensor 11 is any one of a capacitive pressure sensor, a piezoresistive pressure sensor and a piezoelectric thin film sensor, preferably the piezoelectric thin film sensor, and may accurately detect and acquire the pressure signal at each position point. The larger the number of the pressure sensors 11 arranged on the glove body 10, pressure signals at more force points may be acquired, and the more accurate an analysis of the force applied parameters in a process of closed fracture reduction.

In the embodiments of the present disclosure, in order to accurately detect the force applied parameters in the process of closed fracture reduction and avoid an over-complicated data processing, the positions, provided with the pressure sensors 11, on the glove body 10 include a position corresponding to a first position on the palm used to fix the force applied part and a position corresponding to a second position on the palm used to perform traction and diaplasis on the force applied part when the diaplasis is performed by using the glove body.

It should be appreciated that the implementation of closed fracture reduction is mainly divided into two processes. A first process is a traction process. In the process, two doctors stand opposite to each other and fix a proximal end and a distal end of a fracture bone of a patient respectively and perform traction. Taking a Colles' fracture as an example, the doctors fix a wrist joint that is used as a main force applied position of the patient by using medial sides (at hypothenar positions) of two palms and perform traction. In addition, finger pulps of ten fingers are also required to be participated in the fixation of both ends of an affected limb during traction. Therefore, the first position on the palm used to fix the force applied part when the diaplasis is performed by using the glove body includes a hypothenar position on a medial side of the palm and finger pulp positions of five fingers of the palm. A second process is a diaplasis process. In the process, five fingers, a thenar position, a lateral position at a first joint of an index finger close to a thumb, a lateral position at a second joint of the index finger close to the thumb, a position at the first joint of the index finger on a palmar surface and a position at the second joint of the index finger on the palmar surface of each of the doctor's two hands are all required to be participated in an alignment process of two sides of fracture ends in a cooperation manner. Therefore, the second position on the palm used to perform traction and diaplasis on the applied part includes finger pulps of five fingers of the palm, the thenar position of the palm, the lateral position at the first joint of the index finger close to the thumb, the lateral position at the second joint of the index finger close to the thumb, the position at the first joint of the index finger on the palmar surface and the position at the second joint of the index finger on the palmar surface.

Based on the above, according to the embodiments of the present disclosure, the number of the pressure sensors 11 arranged on the glove body 10 is at least 13. As shown in FIG. 2, the positions, provided with the pressure sensors 11, on one glove body 10 include positions corresponding to finger pulp positions 1 of five fingers, a thenar region 2, a hypothenar region 3, a palmar surface 4 at the first joint of the index finger, a lateral surface 5 at the first joint of the index finger, a palmar surface 6 at the second joint of the index finger, and a lateral surface 7 at the second joint of the index finger respectively. The finger pulp positions 1 of the five fingers are each provided with one pressure sensor 11, the thenar region 2 and the hypothenar region 3 are each provided with two pressure sensors 11, and the palmar surface 4 at the first joint of the index finger, the lateral surface 5 at the first joint of the index finger, the palm-facing surface 6 at the second joint of the index finger and the lateral surface 7 at the second joint of the index finger are each provided with one pressure sensor 11.

In the embodiments of the present disclosure, optionally, the number of the glove bodies 10 is two, and the plurality of positions on each glove body 10 is provided with the pressure sensors 11. In one embodiment of the present disclosure, arrangement positions of the pressure sensors 11 on two glove bodies 10 are the same with each other. Optionally, the pressure sensors 11 are arranged according to the arrangement described above.

In the embodiments of the present disclosure, the data collection module 20 and/or the transmission module 30 may be mounted on the glove body 10 and located at a position that does not affect the implementation of closed fracture reduction of the glove body 10, for example, located at a wrist portion of the glove body 10, or located at a position, extending towards an arm portion, of the wrist portion of the glove body 10. In another embodiment of the present disclosure, optionally, the diaplasis detection apparatus further includes a mounting housing 40. As shown in FIG. 1, the mounting housing 40 is separate from the glove body 10, and the data collection module 20 and the transmission module 30 are fixed on the mounting housing 40. In the embodiment of the present disclosure, the data collection module 20 located on the mounting housing 40 may be connected to each pressure sensor 11 on the glove body 10 via a connection line. When the closed fracture reduction is performed, an implementer may wear the glove body 10 on his/her hand, the mounting housing 40 is fixed on the implementer's clothes, and a length of the connection line between the data collection module 20 and the pressure sensor 11 is determined based on that the connection line does not affect the implementation of closed fracture reduction.

In one embodiment of the present disclosure, when the number of the glove bodies 10 is two, optionally, the number of the mounting housings 40 is one, and the data collection module 20 on the mounting housing 40 is connected to each pressure sensor 11 on the two glove bodies 10.

Optionally, when the number of the glove bodies 10 is two, the number of the mounting housings 40 may also be two, each mounting housing 40 is provided with the data collection module 20 and the transmission module 30, and the data collection module 20 on one mounting housing 40 is connected to each pressure sensor 11 on one corresponding glove body 10 via the connection line.

In the embodiments of the present disclosure, optionally, the data collection module 20 includes a plurality of data collection channels, each data collection channel is configured to acquire the pressure signal detected by one corresponding piezoelectric thin film sensor 11, and the transmission module 30 is configured to acquire the pressure signal on each data collection channel and transmit the pressure signal on the data collection channel to the processing apparatus.

Optionally, the data collection module 20 includes a data processing chip having general-purpose input/output (GPIO) ports, and each GPIO port corresponds to one data collection channel.

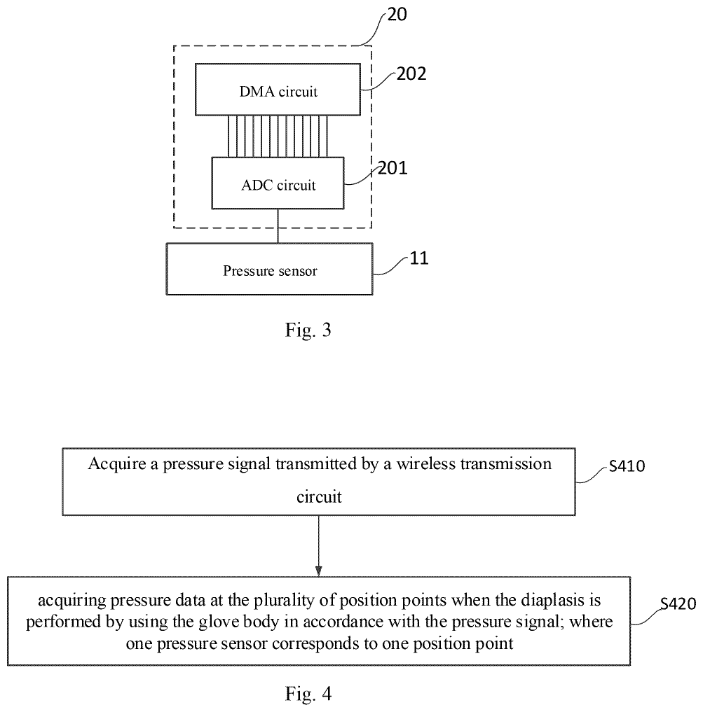

Optionally, the data processing chip of the data collection module 20 is an embedded system STM32 chip having 32 channels and is configured to collect and transmit data of the pressure sensor 11. The STM32 chip includes an analogue-to-digital conversion (ADC) circuit 201 and a direct memory access (DMA) module 202. As shown in FIG. 3, a schematic structural diagram of a connection relationship between the data collection module 20 and the pressure sensor 11 is shown. The data collection module 20 collects the pressure signal on the pressure sensor 11 via the GPIO port of the multipath analogue-to-digital conversion (ADC) circuit 201, the analogue-to-digital conversion (ADC) circuit 201 of each path collects the pressure signal of one pressure sensor 11, directly transmits the collected pressure signal to a serial port via the DMA module 202, and then transmits the pressure signal to the transmission module 30 via the serial port.

In the embodiments of the present disclosure, the data processing chip of the data collection module 20 is the embedded system STM32 chip, and when there are thirteen pressure sensors 11 arranged on the glove body 10, one data collection module 20 may be connected to each pressure sensor 11 on the two glove bodies 10.

Optionally, there may be two data collection modules 20 in the above implementation, and each data collection module 20 includes the STM32 chip. The analog-to-digital conversion (ADC) circuit 201 of each data collection module 20 is connected to the pressure sensors 11 on one glove body 10.

It should be appreciated that the data processing chip of the data collection module 20 is not limited to the embedded system STM32, so long as the number of the data collection channels provided in the selected data processing chip can meet a requirement that the pressure signals on all the pressure sensors 11 may be collected.

In the diaplasis detection apparatus according to the embodiments of the present disclosure, optionally, the transmission module 30 may include, but is not limited to, a Bluetooth transmission module or a Wireless Fidelity (Wi-Fi) transmission module.

In the diaplasis detection apparatus according to the embodiments, the force applied parameters at the plurality of force applied points during the implementation of closed fracture reduction may be collected, so as to facilitate the subsequent case guidance, robot-arm implementation, medical course, etc.

A diaplasis data processing method for performing diaplasis data processing by using the above-mentioned diaplasis detection apparatus is further provided in the embodiment of the present disclosure. As shown in FIG. 4 in combination with FIG. 1 to FIG. 3, the method includes step S410 of acquiring the pressure signal transmitted by the transmission module; and step S420 of acquiring pressure data at the plurality of position points when the diaplasis is performed by using the glove body in accordance with the pressure signal; where one pressure sensor corresponds to one position point.

In the embodiment, optionally, at step S410, when the transmission module 30 is the Bluetooth transmission module, the pressure signal transmitted by the transmission module 30 is acquired through Bluetooth.

Further, when the data collection module includes the plurality of data collection channels, at step S410, the pressure signal of each data collection channel transmitted by the transmission module 30 is acquired, where one data collection channel corresponds to one pressure sensor 11.

Optionally, step S420 of acquiring pressure data at the plurality of position points when the diaplasis is performed by using the glove body in accordance with the pressure signal, includes: caching pressure information about the pressure signal on each data collection channel transmitted by the transmission module 30; optionally, the pressure information is represented by a voltage value; and comparing a voltage value acquired on each data collection channel with a calibration value of the pressure sensor 11 corresponding to the data collection channel, and determining an actual pressure value on each data collection channel, i.e., acquiring the pressure data at each position point.

Optionally, subsequent to the step S420, the diaplasis data processing method further includes establishing an association relationship between the pressure data and pre-stored user information; and storing the pressure data and the association relationship.

Optionally, the pre-stored user information may include, but is not limited to, a name, an age, a body mass index (BMI), a fracture type, a bone density, etc. The user information may be stored as an electronic file.

In the above, the pressure data detected by various pressure sensors 11 that is acquired according to the above method is integrated and stored in the electronic file corresponding to the user information, so as to establish the association relationship with the user information, thereby to facilitate the subsequent data search, case guidance, medical course construction, etc.

In one embodiment of the present disclosure, optionally, subsequent to the step S420, the diaplasis data processing method further includes: receiving a first data retrieval instruction; and displaying pressure data at one of the position points when the diaplasis is performed by using the glove body in response to the first data retrieval instruction.

The one of the position points may be a monitoring point corresponding to any one of the pressure sensors 11. Optionally, in the method according to the embodiment, the pressure data collected by any one of the pressure sensors 11 may be retrieved in real time after the closed fracture reduction has been performed by using the above glove body.

In another embodiment of the present disclosure, optionally, subsequent to the step S420, the diaplasis data processing method further includes displaying a time-changing waveform of the pressure data at one of the position points when the diaplasis is performed by using the glove body.

The one of the position points may be the monitoring point corresponding to any one of the pressure sensors 11. The time-changing waveform of the pressure data at the one of the position points may be displayed in real time during the implementation of closed fracture reduction or may be displayed after the pressure data associated with the user information is retrieved subsequent to the implementation of closed fracture reduction.

Optionally, after the implementation of closed fracture reduction, a second data retrieval instruction may be inputted by a user, and the time-changing waveform of the pressure data at one of the position points is displayed in accordance with an indication in the second data retrieval instruction.

In another embodiment of the present disclosure, optionally, the diaplasis data processing method further includes displaying a magnitude relationship among the pressure data at the position points in a comparison manner when the diaplasis is performed by using the glove body.

Optionally, the magnitude relationship among the pressure data at the position points may be displayed in the comparison manner during or after the implementation of closed fracture reduction.

Optionally, after the implementation of closed fracture reduction, a third data retrieval instruction may be inputted by the user, and the magnitude relationship among the pressure data at the position points during the implementation of closed fracture reduction is displayed in the comparison manner in accordance with the third data retrieval instruction.

Specifically, a plurality of region patterns may be displayed on a display interface, the plurality of region patterns correspond to the plurality of pressure sensors 11 respectively, and each region pattern is configured to display the pressure data level of one pressure sensor 11. The pressure data detected by the pressure sensor 11 may be displayed in different colors and/or brightness values according to different pressure levels, so as to distinguish the different pressure levels, and compare levels of the pressure data at the position points corresponding to different pressure sensors 11 visually. For example, when the pressure data is located in different pressure ranges, the region patterns are displayed in different colors, and within a same pressure range, the corresponding displayed color may be gradually darkened with the gradual increase of the pressure data.

In the diaplasis data processing method implemented by using the diaplasis detection apparatus according to the embodiments of the present disclosure, the force applied parameters at the plurality of force applied points during the implementation of closed fracture reduction may be associated and stored with the corresponding user information, the force applied parameters may be further retrieved in real time, and values of the force applied parameters at various force applied points may be displayed and compared with each other, so as to facilitate the subsequent case guidance, robot-arm performing, medical course, etc.

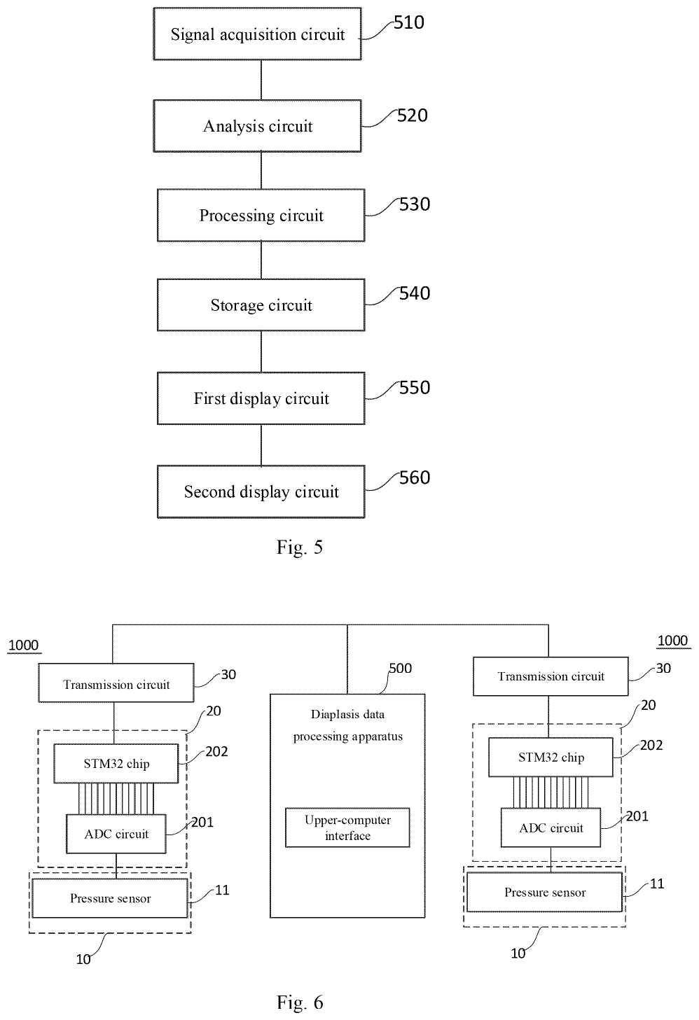

In another aspect, a diaplasis data processing apparatus is further provided in the embodiments of the present disclosure. As shown in FIG. 5, the diaplasis data processing apparatus 500 for performing diaplasis data processing by using the above-mentioned diaplasis detection apparatus includes: a signal acquisition module 510, configured to acquire the pressure signal transmitted by the transmission module; and an analysis module 520, configured to acquire pressure data at the plurality of position points when the diaplasis is performed by using the glove body in accordance with the pressure signal; where one pressure sensor corresponds to one position point.

Optionally, as shown in FIG. 5, the diaplasis data processing apparatus further includes: a processing module 530, configured to establish an association relationship the pressure data and pre-stored user information; and a storage module 540, configured to store the pressure data and the association relationship.

Optionally, as shown in FIG. 5, the diaplasis data processing apparatus further includes a first display module 550, configured to display a time-changing waveform of the pressure data at one of the position points when the diaplasis is performed by using the glove body.

Optionally, as shown in FIG. 5, the diaplasis data processing apparatus further includes a second display module 560, configured to display a magnitude relationship among the pressure data at the position points in a comparison manner when the diaplasis is performed by using the glove body.

In another aspect, a diaplasis detection system is further provided in the embodiments of the present disclosure, as shown in FIG. 6 in combination with FIG. 5, the diaplasis detection system includes the above-mentioned diaplasis detection apparatus 1000 and the above-mentioned diaplasis data processing apparatus 500.

In one embodiment of the present disclosure, the diaplasis data processing apparatus 500 is an integrated machine having a data processing function and a display function, and includes the signal acquisition module 510, the analysis module 520, the processing module 530, the storage module 540, the first display module 550 and the second display module 560 which are configured to realize the above functions.

Further, optionally, the diaplasis data processing apparatus 500 further includes a wireless transmission module configured to receive the pressure signal transmitted by the transmission module 30 of the diaplasis detection apparatus 1000.

With reference to FIG. 6, taking two glove bodies 10 which are correspond to left and right hands of a closed fracture diaplasis implementer respectively as an example, each of the two glove bodies 10 is provided with the plurality of pressure sensors 11, and each glove body 10 is connected to one data collection module 20 and one transmission module 30. The pressure sensors 11 on each glove body 10 may collect the pressure signals on the pressure sensors 11 via the GPIO ports of the analogue-to-digital conversion (ADC) circuit 201, and the analogue-to-digital conversion (ADC) circuit 201 of each path collects the pressure signal of one piezoelectric film sensor 11, and directly transmits the collected pressure signal to the serial port of the STM32 chip via the DMA module 202, and then transmits the pressure signal to the transmission module 30 via the serial port.

Further, the transmission modules 30 of the two glove bodies 10 transmit the pressure signal on each data collection channel sequentially to the diaplasis data processing device 500 through wireless transmission.

Optionally, the signal acquisition module 510 of the diaplasis data processing apparatus 500 may perform channel real-time switching on the data collection channels, so as to acquire the pressure signal on each data transmission channel, and the analysis module 520 may cache the pressure information about the pressure signal on each data transmission channel and compare the voltage value acquired by each data collection channel with a calibration value of the pressure sensor 11 corresponding to the data collection channel, and determine an actual pressure value on each data collection channel, i.e., acquire the pressure data at each position point.

Further, in the diaplasis data processing apparatus 500, the processing module 530 may pre-store the user information, establish an association relationship between the acquired pressure data and pre-stored user information, and store the association relationship, to form an electronic medical file including closed fracture reduction data.

Based on the above, the diaplasis data processing apparatus 500 may further include an upper-computer interface capable of receiving a data retrieval instruction from the user, and displaying the pressure data at each position point when the diaplasis is performed by using the glove body, the time-changing waveform of pressure data at one of the position points when the diaplasis is performed by using the glove body, and the magnitude relationship among the pressure data at the position points when the diaplasis is performed by using the glove body, etc. in real time.

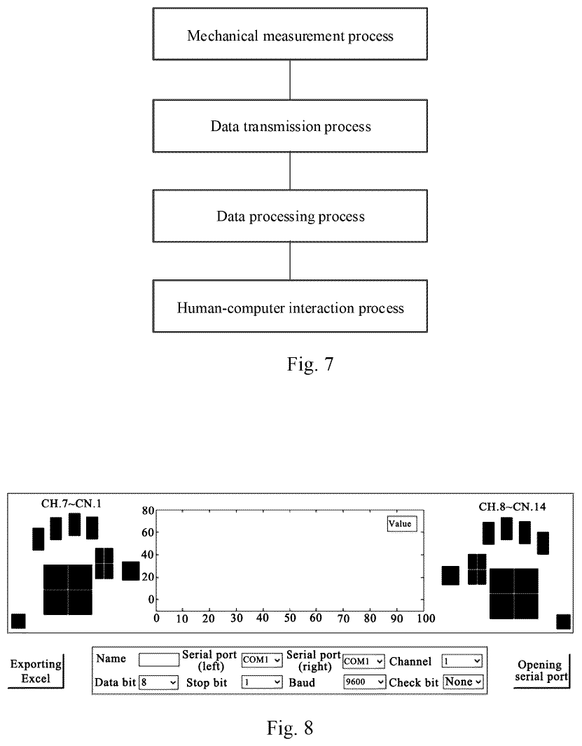

Based on the above, the diaplasis detection system according to the embodiments of the present disclosure, as shown in FIG. 7, mainly includes several main processes of a mechanical measurement process, a data transmission process, a data processing process and a human-computer interaction process.

In the mechanical measurement process, the collection channel of the STM32 chip collects the pressure signal of the piezoelectric thin film sensor on the glove body, the pressure signal is transmitted to the serial port of the STM32 chip via the DMA in real time, and then is transmitted to the diaplasis data processing device 500 by a Bluetooth device connected to the serial port.

In the data transmission process, the diaplasis data processing apparatus 500 acquires pressure data of 26 data collection channels of the STM32 chip via MATLAB or a GUI universal synchronous/asynchronous receiver/transmitter (USART) serial port through Bluetooth, and caches the pressure data.

In the data processing process, the diaplasis data processing apparatus performs on-line filtering processing on the pressure data of the 26 data collection channels acquired in real time, and performs calibration on the pressure data using calibration values of various pressure sensors to acquire an actual pressure value on each data collection channel corresponding to each pressure sensor.

In the human-computer interaction process, the pressure data on any one of the data collection channels (26 data collection channels are provided in the present embodiment) may be retrieved, time-changing waveforms of the pressure data on various data collection channels are displayed in real time, and the magnitude relationship among the pressure data on various data collection channels is displayed in the comparison manner. In accordance with matched user information (including a name, an age, a bone density, a BMI, a fracture type, etc.), the pressure data on the 26 data collection channels throughout the performing of closed fracture diaplasis is associated with the user information to establish the user's electronic medical information.

Optionally, in the human-computer interaction process, a display state of the upper-computer interface is shown in FIG. 8. During and after the implementation of closed fracture reduction, the user information may be inputted on the upper-computer interface, and a pull-down menu of “Channel Number” is selected to select a channel to be viewed, a time-changing pressure data waveform at a corresponding data collection channel during the implementation of fracture reduction may be displayed in a rectangular box in the center, and a specific value of the pressure data may be displayed at “Value” in an upper right position of the rectangular box.

Further, on the upper-computer interface, a group of region patterns, corresponding to the glove bodies of the left and right hands, may be displayed on two sides of the central rectangular box, the plurality of region patterns correspond to the plurality of pressure sensors 11 respectively, and each region pattern is configured to display the pressure data level of one pressure sensor 11. Optionally, the plurality of region patterns are arranged according to positions of the corresponding pressure sensors 11 on the glove body, so the pressure sensor 11 represented by each region pattern may be visually displayed. Specifically, the pressure data detected by the pressure sensors 11 may be distinguished by displaying the region patterns in different colors and/or brightness values according to different pressure levels, and the pressure data levels at the position points corresponding to different pressure sensors 11 may be visually compared. For example, that when the pressure data is located in different pressure ranges, the region patterns are displayed in different colors, and within the same pressure range, the corresponding displayed color may be gradually darkened with the gradual increase of the pressure data.

Optionally, after the data collection in the process of closed fracture reduction is finished, an “Exporting Excel” touch button on the upper-computer interface is clicked, and then current user information and the pressure data collected by each channel are associated and stored as an electronic medical record.

In the diaplasis detection system according to the embodiments of the present disclosure, the pressure data of at least 26 force points on two glove bodies during the implementation of closed fracture reduction may be collected and recorded, and in cooperation with the UI interface of the upper-computer, the change of pressure on each channel may be displayed online and in real time. In addition, basic information about the patient may be integrated to generate the medical record of closed fracture of the patient, so as to provide a quantitative technical solution for teaching, research, and clinical use of the closed fracture reduction.

The above embodiments are for illustrative purposes only, but the present disclosure is not limited thereto. Obviously, a person skilled in the art may make various improvements and modifications without departing from the principle of the present disclosure, and these improvements and modifications shall fall within the scope of protection of the present disclosure.

What is claimed is:

1. A diaplasis detection apparatus, comprising:
a glove body capable of being worn on a hand of a force applying person, wherein a plurality of positions on the glove body is provided with pressure sensors respectively, and the plurality of positions comprises positions on the glove body corresponding to a position on a palm used to fix a force applied part and a position on the palm used to perform traction and diaplasis on the force applied part when the diaplasis is performed by using the glove body;
a data collection module connected to each pressure sensor and configured to collect a pressure signal detected by the pressure sensor; and
a transmission module configured to acquire the pressure signal and transmit the pressure signal to a processing apparatus.

2. The diaplasis detection apparatus according to claim 1, further comprising a mounting housing separate from the glove body, and the data collection module and the transmission module are fixed on the mounting housing.

3. The diaplasis detection apparatus according to claim 1, wherein the plurality of positions comprises the positions on the glove body corresponding to a hypothenar position on a medial side of the palm, finger pulps of five fingers of the palm, a thenar position on the palm, a lateral position at a first joint of an index finger close to a thumb, a lateral position at a second joint of the index finger close to the thumb, a position at the first joint of the index finger on a palmar surface and a position at the second joint of the index finger on the palmar surface.

4. The diaplasis detection apparatus according to claim 3, wherein the data collection module comprises a plurality of data collection channels, each data collection channel is configured to acquire the pressure signal detected by one corresponding pressure sensor, and the transmission module is configured to acquire the pressure signal on each data collection channel and transmit the pressure signal on the data collection channel to the processing apparatus.

5. The diaplasis detection apparatus according to claim 4, wherein the data collection module comprises a data processing chip having general-purpose input/output (GPIO) ports, and each GPIO port corresponds to one data collection channel.

6. The diaplasis detection apparatus according to any one of claims 1 to 5, wherein the transmission module comprises a Bluetooth transmission module or a Wireless Fidelity (Wi-Fi) transmission module.

7. The diaplasis detection apparatus according to any one of claims 1 to 5, wherein the number of glove bodies is two, the plurality of positions on each glove body is provided with the pressure sensors respectively, and the data collection module is connected to each pressure sensor on the two glove bodies.

8. A diaplasis data processing method for performing diaplasis data processing by using the diaplasis detection apparatus according to any one of claims 1 to 7, comprising:
acquiring the pressure signal transmitted by the transmission module; and
acquiring pressure data at the plurality of position points when the diaplasis is performed by using the glove body in accordance with the pressure signal;
wherein one pressure sensor corresponds to one position point.

9. The diaplasis data processing method according to claim 8, further comprising:
establishing an association relationship between the pressure data and pre-stored user information; and
storing the pressure data and the association relationship.

10. The diaplasis data processing method according to claim 8, further comprising:
displaying a time-changing waveform of the pressure data at one of the position point when the diaplasis is performed by using the glove body.

11. The diaplasis data processing method according to claim 8, further comprising:
displaying a magnitude relationship among the pressure data at the position points in a comparison manner when the diaplasis is performed by using the glove body.

12. A diaplasis data processing apparatus for performing diaplasis data processing by using the diaplasis detection apparatus according to any one of claims 1 to 7, comprising:
a signal acquisition module, configured to acquire the pressure signal transmitted by the transmission module; and
an analysis module, configured to acquire pressure data at the plurality of position points when the diaplasis is performed by using the glove body in accordance with the pressure signal;
wherein one pressure sensor corresponds to one position point.

13. The diaplasis data processing apparatus according to claim 12, further comprising:
a processing module, configured to establish an association relationship between the pressure data and pre-stored user information; and
a storage module, configured to store the pressure data and the association relationship.

14. The diaplasis data processing apparatus according to claim 12, further comprising:
a first display module, configured to display a time-changing waveform of the pressure data at one of the position points when the diaplasis is performed by using the glove body.

15. The diaplasis data processing apparatus according to claim 12, further comprising:
a second display module, configured to display a magnitude relationship among the pressure data at the position points in a comparison manner when the diaplasis is performed by using the glove body.

16. A diaplasis detection system comprising the diaplasis detection apparatus according to any one of claims 1 to 7 and the diaplasis data processing apparatus according to any one of claims 12 to 15.
